(19) United States
(12) Patent Application Publication
(10) Pub. No.: US 2009/0292144 A1
(43) Pub. Date: Nov. 26, 2009

(54) DIRECT AMINATION OF HYDROCARBONS

(75) Inventors: Joachim-Thierry Anders, Goennheim (DE); Johann-Peter Melder, Boehl-Iggelheim (DE); Petr Kubanek, Mannheim (DE); Ekkehard Schwab, Neustadt (DE); Wolfgang Mackenroth, Tervuren (BE); Karl Hoelemann, Mannheim (DE); Frederik Van Laar, Dubai (AE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, LUDWIGSHAFEN (DE)

(21) Appl. No.: 12/373,056

(22) PCT Filed: Jul. 17, 2007
(86) PCT No.: PCT/EP07/57355

§ 371 Date: Jan. 9, 2009

(30) Foreign Application Priority Data
Jul. 21, 2006 (EP) 06117623.6

(51) Int. Cl. C07C 209/00

(52) U.S. Cl. 564/408

(57) ABSTRACT

A process for aminating hydrocarbons with ammonia, which comprises performing the amination in the presence of an additive which reacts with hydrogen, the additive used being at least one organic chemical compound, N₂O, hydroxylamine, hydrazine and/or carbon monoxide.

The invention relates to a process for preferably continuous amination, preferably direct amination of hydrocarbons, preferably by reacting hydrocarbons, more preferably aromatic hydrocarbons, especially benzene, with ammonia, preferably in the presence of catalysts which catalyze the amination, the amination being performed in the presence of an additive which reacts with hydrogen, the additive used being at least one organic chemical compound, N₂O, hydroxylamine, hydrazine and/or carbon monoxide. In this document, the expression “additive” should be understood to mean one or more additives which react(s) with hydrogen. The expression “additive which reacts with hydrogen” should be understood hereinafter to mean both organic chemical compounds and carbon monoxide. The additive is preferably nitrobenzene.

In particular, the invention relates to processes for aminating hydrocarbons, preferably by reacting aromatic hydrocarbons, more preferably benzene, with ammonia, especially according to the following reaction which is preferably catalyzed:

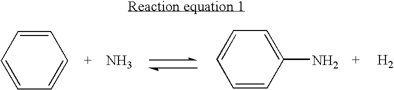

The commercial preparation of amines, especially of aromatic amines such as aniline, is typically performed in multistage reactions. Aniline is prepared, for example, typically by converting benzene to a benzene derivative, for example nitrobenzene, chlorobenzene or phenol, and then converting this derivative to aniline.

More advantageous than such indirect processes for preparing especially aromatic amines are methods which enable direct preparation of the amines from the corresponding hydrocarbons. A very elegant route is the heterogeneously catalyzed direct amination of benzene, described for the first time in 1917 by Wibaut (Berichte, 50, 541-546). Since direct amination is equilibrium-limited, several systems have been described which shift the equilibrium limit by the selective removal of hydrogen from the reaction and enable increased benzene conversion. Most processes are based on the use of metal oxides which are reduced by hydrogen, thus removing hydrogen from the reaction system and hence shifting the equilibrium.

CN 1555921A discloses the oxidoamination of benzene in the liquid phase, hydrogen peroxide functioning as the “O” donor. However, the use of H₂O₂ is suitable only to a limited degree for bulk chemicals owing to the cost and the low selectivity owing to subsequent reactions.

CA 553,988 discloses a process for preparing aniline from benzene, in which benzene, ammonia and gaseous oxygen are reacted over a platinum catalyst at a temperature of about 1000° C. Suitable platinum-comprising catalysts are platinum alone, platinum with certain specific metals and platinum together with certain specific metal oxides. In addition, CA 553,988 discloses a process for preparing aniline, in which benzene in the gas phase is reacted with ammonia in the presence of a reducible metal oxide at temperatures of from 100 to 1000° C. without addition of gaseous oxygen. Suitable reducible metal oxides are the oxides of iron, nickel, cobalt, tin, antimony, bismuth and copper.

U.S. Pat. No. 3,919,155 relates to the direct amination of aromatic hydrocarbons with ammonia, in which the catalyst used is nickel/nickel oxide, and the catalyst may additionally comprise oxides and carbonates of zirconium, strontium, barium, calcium, magnesium, zinc, iron, titanium, aluminum, silicon, cerium, thorium, uranium and alkali metals.

U.S. Pat. No. 3,929,889 likewise relates to the direct amination of aromatic hydrocarbons with ammonia over a nickel/nickel oxide catalyst, the catalysts used having been partly reduced to elemental nickel and subsequently reoxidized to obtain a catalyst which has a ratio of nickel:nickel oxide of from 0.001:1 to 10:1.

U.S. Pat. No. 4,001,260 relates to a process for the direct amination of aromatic hydrocarbons with ammonia, in which a nickel/nickel oxide catalyst is again used, which is applied to zirconium dioxide and has been reduced with ammonia before use in the amination reaction.

U.S. Pat. No. 4,031,106 relates again to the direct amination of aromatic hydrocarbons with ammonia over a nickel/nickel oxide catalyst on a zirconium dioxide support which further comprises an oxide selected from lanthanoids and rare earth metals.

DE 196 34 110 describes nonoxidative amination at a pressure of 10-500 bar and a temperature of 50-900° C., the reaction being effected in the presence of an acidic heterogeneous catalyst which has been modified with light and heavy platinum group metals.

WO 00/09473 describes to a process for preparing amines by direct amination of aromatic hydrocarbons over a catalyst comprising at least one vanadium oxide.

WO 99/10311 relates to a process for the direct amination of aromatic hydrocarbons at a temperature of <500° C. and a pressure of <10 bar. The catalyst used is a catalyst comprising at least one metal selected from transition metals, lanthanides and actinides, preferably Cu, Pt, V, Rh and Pd. Preference is given to carrying out the direct amination in the presence of an oxidizing agent to increase the selectivity and/or the conversion. The oxidizing agent is preferably an oxygen-comprising gas, for example air, O₂-enriched air, O₂/inert gas mixtures or pure oxygen.

WO 00/69804 relates to a process for the direct amination of aromatic hydrocarbons, in which the catalyst used is a complex comprising a noble metal and a reducible metal oxide. Particular preference is given to catalysts comprising palladium and nickel oxide or palladium and cobalt oxide.

Indirect syntheses are also disclosed in CN 1424304, CN 1458140 and WO 2004/052833.

Most of the processes mentioned start from a mechanism for direct amination as detailed in the abstract of WO 00/69804. According to this, the desired amine compound is initially prepared under (noble) metal catalysis from the aromatic hydrocarbon and ammonia, and the hydrogen formed in the first step is “scavenged” in a second step with a reducible metal oxide. The same mechanistic considerations form the basis of the process in WO 00/09473, in which the hydrogen is scavenged with oxygen from vanadium oxides (page 1, lines 30 to 33). The same mechanism also forms the basis in U.S. Pat. No. 4,001,260, as is evident from the remarks and the diagram in column 2, lines 16 to 44.

It is an object of the present invention to develop a particularly economically viable process for aminating hydrocarbons, especially a process for reacting benzene with ammonia, in which a preferably continuous process with very high selectivity and/or very high conversion is enabled.

This object is achieved by the process detailed at the outset.

It has been found that, surprisingly, addition of an organic chemical substance which reacts with hydrogen, and/or carbon monoxide, preferably into the feed, increased the conversion to the product of value with the same selectivity.

For example, the direct amination of benzene with ammonia (according to reaction equation 1 on page 1) forms ammonia, but one mole of hydrogen is also formed at the same time. Moreover, hydrogen may also be present in the reaction vessel as a result of the decomposition of ammonia. According to the technical teachings of the prior art, ammonia is significantly decomposed to give hydrogen and nitrogen, for example by the nickel-nickel oxide systems.

Irrespective of which source the hydrogen stems from, it limits the conversion of the direct amination reaction. Since the reaction shown in reaction equation 1 is an equilibrium reaction, the quotient of the product of the concentrations or partial pressures of the products and those of the reactants is a constant; see physical chemistry textbooks: Peter Atkins; Julio de Paula, Atkins' Physical Chemistry, 8^{th} edition, Oxford: Oxford University Press, 2006, ISBN 0-19-870072-5 or Gerd Wedler, Lehrbuch der physikalischen Chemie [Textbook of physical chemistry], 5th, fully revised and updated edition, Weinheim: Wiley-VCH, 2004, ISBN 3-527-31066-5). A high concentration of hydrogen therefore brings about a lower conversion of benzene to aniline. Conversely, especially hydrogen which gets into the reaction system additionally, for example from the decomposition of ammonia, can influence the equilibrium of the reaction and force it back to the reactant side, i.e. even bring about dissociation of aniline into the benzene and ammonia reactants.

It is therefore advantageous to minimize the hydrogen concentration in the reaction system.

It is particularly advantageous when the hydrogen concentration is minimized, instead of the metered addition of oxygen (WO 99/10311) or hydrogen peroxide (CN 1555921) cited in the earlier prior art, by selecting one or more organic chemical substance(s) and/or carbon monoxide as an additive. In particular, it is advantageous to select specifically that organic chemical substance which, on reaction with the hydrogen present in the system, simultaneously forms the same reaction product which is also formed in the direct amination reaction.

The process according to the invention removes hydrogen, both from the direct amination reaction and from the ammonia decomposition, from the reaction system, and reduces or prevents the forcing of the equilibrium back to the side of the reactants, i.e. the reduction in the content of hydrogen in the equilibrium even increases the conversion of the direct amination reaction. The lowering of the hydrogen concentration in the reaction mixture has a direct influence on the conversion to the product of value, since the direct amination is an equilibrium reaction.

In a particularly preferred embodiment of the process, the hydrogen is utilized productively by generating additional product of value by virtue of the hydrogenation of the additive into the feed. The reaction of the hydrogen with the organic chemical additive, where it is an additive which reacts with hydrogen to give the same product as the hydrocarbon in the direct amination, also does not introduce an extraneous product in a coproduction—this means that the removal of the hydrogen scavenging product from the direct amination product is also dispensed with and the complexity for the workup of the reaction product is therefore significantly reduced. This very elegant solution does not only shift the equilibrium, it additionally utilizes the undesired by-product for the preparation of the desired product of value.

In a further preferred embodiment of the process, the organic chemical additive reacts with hydrogen to give one of the reactants.

The metered addition of organic chemical additives is advantageous over the prior art. In most of the abovementioned documents, metal oxides are used as catalysts or cataloreactants. When only the oxygen from these catalysts or cataloreactants is to remove the hydrogen from the system, this implicitly entails the disadvantage that the catalysts or cataloreactants deactivate rapidly because the oxygen present is depleted by reduction both by the hydrogen formed in the direct amination reaction and by the hydrogen released in the ammonia dissociation. In that case, more metal centers of the (0) oxidation state are additionally present in the reduced catalysts or cataloreactants, which, as experience has shown, enhance the decomposition of ammonia even further, which even further accelerates the deactivation of the catalysts or cataloreactants and entails earlier regeneration of the catalysts or cataloreactants—with corresponding effects on the economic viability of the process. The addition of oxygen is also inferior to the use of organic chemical substances, because it can result in total combustion of the organic ingredients of the reaction system to CO₂ in a considerable degree on the catalytically active metal surfaces, again with considerable effects on the economic viability of the process. Owing to relatively low selectivity, the addition of hydrogen peroxide is at a disadvantage compared to the process according to the invention.

All of these disadvantages can be overcome by the process according to the invention without any need to significantly change pressure or temperature.

The additives used in accordance with the invention may react with hydrogen. They are preferably organic chemical substances which can react with hydrogen. They are more preferably organic chemical substances which, in the reaction with hydrogen, form the same reaction product which is also formed in the direct amination of hydrocarbons.

Particular preference is given to the direct amination of benzene with ammonia to give aniline; the particularly preferred organic chemical additive likewise forms aniline in the reaction with hydrogen; the organic chemical additive is more preferably nitrobenzene.

In a further preferred embodiment of the invention, the organic chemical additive used in the direct amination of hydrocarbons, preferably the direct amination of benzene with ammonia to give aniline, is N₂O, hydroxylamine and/or hydrazine.

The advantage that the product of value is formed when nitrobenzene is used and reacts with hydrogen does not occur when hydroxylamine or hydrazine are used as hydrogen scavengers. On the other hand, however, when hydroxylamine or hydrazine are used as the hydrogen scavenger, the reaction with hydrogen releases ammonia, and thus one of the reactants, which is equally preferred because the formation of aniline is promoted thermodynamically in the equilibrium when the ammonia excess is increased; in addition, the equilibrium is shifted toward aniline when the hydrogen content falls.

The organic chemical additives which react with hydrogen may—but need not exclusively—be oxidizing agents known as such. Instead, useful organic chemical additives also include all molecules with reducible functionalities, especially those which comprise multiple bonds. These molecules or the products of their reaction with hydrogen should preferably not enter into a direct reaction with the hydrocarbon because this would impair the selectivity of the direct amination.

In addition to nitrobenzene, useful compounds for use in the process according to the invention are, for example, carbon monoxide, carbonyl compounds, nitriles, imines, amides, nitro compounds, nitroso compounds, olefins, alkynes, organic peroxides, organic acids, organic acid derivatives, hydrazine derivatives, hydroxylamines, quinones, aromatics and/or molecules with sp2-hybridized carbon atoms, and also all further molecules with reducible functionalities, especially those which comprise multiple bonds, or combinations thereof.

Specific examples (by way of example without restricting the scope of the invention to these molecules) of inventive organic chemical additives selected from the abovementioned substance groups include nitrobenzene, carbon monoxide, hydrocyanic acid, acetonitrile, propionitrile, butyronitrile, benzonitrile, imines from the reaction of benzaldehyde with ammonia or primary amines, imines from the reaction of aliphatic aldehydes with ammonia or primary amines, formamide, acetamide, benzamide, nitrosobenzene, ethene, propene, 1-butene, 2-butene, isobutene, n-pentene and pentene isomers, cyclopentene, n-hexene, hexene isomers, cyclohexene, n-heptene, heptene isomers, cycloheptene, n-octene, octadienes, cyclooctene, cyclooctadiene, acetylene, propyne, butyne, phenylacetylene, meta-chloroperbenzoic acid, formic acid, acetic acid, propionic acid, butyric acid, valeric acid, caproic acid, oxalic acid, malonic acid, succinic acid, maleic acid, fumaric acid, esters or anhydrides of the carboxylic acids mentioned, hydrazine, phenylhydrazine, hydrazides of aliphatic or aromatic ketones, hydroxylamine, alkylhydroxylamines and arylhydroxylamines (or combinations of the substances mentioned).

Particular preference is given in particular to using for this purpose reducible nitrogen compounds such as nitriles, nitro compounds, nitroso compounds and amides, and also acetylene and short-chain alkynes, preferably having from 3 to 6 carbon atoms, and also short-chain olefins, preferably having from 2 to 6 carbon atoms, or combinations thereof.

With very particular preference, nitrobenzene, nitrosobenzene, carbon monoxide, acetylene, ethene, propene, hydrazine, phenylhydrazine, hydroxylamine, phenylhydroxylamine, acetonitrile, benzonitrile or combinations thereof may be selected as organic chemical additives for the process according to the invention.
The additive which reacts with hydrogen can be metered in at any point in the process. It is possible, for example, to separately feed hydrocarbon, preferably benzene, amine, preferably ammonia, and organic chemical additive, preferably nitrobenzene, into the reactor,
the combined feeding of hydrocarbon, preferably benzene, and organic chemical additive, preferably nitrobenzene, in a common feed line, separately from the amine, preferably ammonia, into the reactor,
the combined feeding of amine, preferably ammonia, and organic chemical additive, preferably nitrobenzene, in a common feed line, separately from the hydrocarbon, preferably benzene, into the reactor,
the feeding of the individual components at different points in the reactor (for example one or more of the three components at the reactor inlet, one just above or into the catalyst bed).

Preference is given to the metered addition of the additive which reacts with hydrogen together with the hydrocarbon, preferably benzene, at the inlet of the reactor. Very particular preference is given to the metered addition of a nitrobenzene/benzene mixture in a common feed line and ammonia in another feed line, in each case at the inlet of the reactor. It is likewise very preferred to combine the metered addition of a nitrobenzene/benzene mixture in a common feed line and the metered addition of the ammonia from a second feed line initially in a mixer or evaporator in order to feed a homogeneous mixture to the catalyst bed.

The molar hydrocarbon/organic chemical additive ratio may be selected within a very wide range, since even small additions have an effect but even relatively high additions are not harmful. The molar ratio of hydrocarbon to organic chemical additive can thus be varied within a range of from 10 000:1 to 1:1000.

However, it is advantageous to add a relatively small amount of the hydrogen scavenger, for example between 0.001% by weight and 50% by weight, based on the total weight of the hydrocarbon used and the additive which reacts with hydrogen.

The proportion by weight of the additive which reacts with hydrogen is thus more preferably between 0.001% by weight and 50% by weight, in particular between 0.1% by weight and 15% by weight, most preferably between 0.5% by weight and 3% by weight, based in each case on the total weight of the hydrocarbon used, preferably benzene, and the additive, preferably nitrobenzene, a mixture of benzene and nitrobenzene preferably being used as the aromatics feed in the process for the direct amination of benzene.

When hydroxylamine or hydrazine is used as the hydrogen scavenger, it is also possible to proceed analogously to the method in the case of nitrobenzene. The abovementioned preferred proportions by weight, based on the benzene feed, also apply preferentially to these substances.

All remaining reaction conditions may be selected in accordance with the prior art. Preference is given to working at temperatures between 300° C. and 500° C., more preferably between 350 and 400° C. The reaction pressure is typically between 1 and 1000 bar, preferably between 2 and 300 bar, more preferably between 2 and 150 bar.

It is thus a further advantage of the process according to the invention that, in comparison to working without an organic chemical additive (including hydroxylamine, N₂O, hydrazine and carbon monoxide), no changes regarding the reaction conditions of the direct amination are required.

The catalysts used may be the catalysts known for the direct amination of hydrocarbons, especially those known for the direct amination of benzene with ammonia to give aniline. Such catalysts have been described in a wide variety in the patent literature and are commonly known. Useful catalysts include, for example, customary metal catalysts, for example those based on nickel, iron, cobalt, copper, noble metals or alloys of these metals mentioned. Useful noble metals (NM) may include all noble metals, for example Ru, Rh, Pd, Ag, Ir, Pt and Au, the noble metals Ru and Rh preferably not being used alone but rather in alloy with other transition metals, for example Co, Cu, Fe and nickel or mixtures thereof. Such alloys are also used with preference in the case of use of the other noble metals; for example, supported NiCuNM; CoCuNM; NiCoCuNM, NiMoNM, NiCrNM, NiReNM, CoMoNM, CoCrNM, CoReNM, FeCuNM, FeCoCuNM, FeMoNM, FeReNM alloys are of interest, where NM is a noble metal, especially preferably Ag and/or Au.

The catalyst may be used in generally customary form, for example as a powder or as a system usable in a fixed bed (for example extrudates, spheres, tablets, rings), in which case the catalytically active constituents may, if appropriate, be present on a support material. Useful support materials include, for example, inorganic oxides, for example ZrO₂, SiO₂, Al₂O₃, TiO₂, B₂O₃, ThO₂, CeO₂, Y₂O₃ and mixtures of these oxides, preferably TiO₂, ZrO₂, Al₂O₃ and SiO₂, more preferably ZrO₂. ZrO₂ is understood to mean either pure ZrO₂ or the normal Hf-comprising ZrO₂.

The catalyst more preferably catalyzes both the direct amination of the hydrocarbons and the hydrogenation of the organic chemical additive (including carbon monoxide), so that no further catalyst is required for the hydrogenation of the additive.

The catalysts used with preference in the process according to the invention may be regenerated, for example by passing a reductive atmosphere (for example H₂ atmosphere) over the catalyst or first an oxidative and then a reductive atmosphere over or through the catalyst bed.

The catalyst may be present either in its reduced or oxidized form; it is preferably present in its oxidized form.

The catalyst used is preferably a compound which comprises one or more elements selected from the group of Ni, Cu, Fe, Co, preferably in combination with Mo or Ag, where the elements may each be present in reduced and/or oxidized form. Particularly preferred catalysts are the combinations Co—Cu, Ni—Cu and/or Fe—Cu, especially the combinations thereof with an additional doping element Ni—Cu—X, Fe—Cu—X, Co—Cu—X where X is Ag or Mo. Especially preferred are alloys of NiCu(Ag or Mo) and/or FeCu(Ag or Mo).

In the catalyst, the proportion by weight of the elements Ni, Co and Fe together, i.e. the proportion of the total weight of these elements, not all elements necessarily being present in the catalyst, is preferably between 0.1% by weight and 75% by weight, more preferably between 1% by weight and 70% by weight, in particular between 2% by weight and 50% by weight, and the proportion by weight of Cu is between 0.1% by weight and 75% by weight, preferably between 0.1% by weight and 25% by weight, more preferably between 0.1% by weight and 20% by weight, in particular between 2.5% by weight and 10% by weight, based on the total weight of catalyst. In addition, the catalyst may comprise support material.

The proportion by weight of the doping element X in the total weight of catalyst is preferably between 0.01% by weight and 8% by weight, more preferably between 0.1% by weight and 5% by weight, in particular between 0.5% by weight and 4% by weight.

The catalyst can preferably be activated before use in the process. Such an activation, which is preferably effected at a temperature between 200 and 600° C., more preferably at temperatures between 250 and 500° C., in particular at temperatures between 280 and 400° C., is preferably carried out with a mixture comprising inert gas and hydrogen or ammonia. The activation gas may also comprise further compounds. The activation reduces the metal oxides to the metal.

In addition, the catalysts used may be compounds which comprise Cu, Fe, Ni or mixtures thereof, which are supported on layered double hydroxides (LDH) or LDH-like compounds. Preference is given to using magnesium aluminum oxide, which is obtainable by calcining LDH or LDH-like compounds, as the support. A suitable process for preparing magnesium aluminum oxide, comprising the step of calcining LDH or LDH-like compounds, is disclosed, for example, in Catal. Today 1991, 11, 173 or in “Comprehensive Supramolecular Chemistry”, (Ed. Alberti, Bein), Pergamon, N.Y., 1996, Vol 7, 251.

In one embodiment of the process according to the invention, the catalyst used is more preferably a compound which comprises one or more compounds selected from the group of Ni, Cu, Co, Fe and Mo, and these elements may be present in one or more oxidation states, preferably on zirconium oxide and/or magnesium aluminum oxide as the support.

In this embodiment, the catalyst used is most preferably at least one of the following compounds (a), (b), (c) and/or (d):
(a) comprising, preferably consisting of, NiO, CuO and MoO₃, on zirconium oxide as a support,
(b) (preferably mainly) comprising NiO, CuO and CoO on zirconium oxide as a support,
(c) comprising, preferably consisting of, NiO and/or CuO and/or Fe₂O₃ on zirconium oxide as a support,
(d) comprising, preferably consisting of, NiO and/or CuO and/or Fe₂O₃ on magnesium aluminum oxide as a support,
   catalysts (a) to (d) being usable individually or in combination with one another.

The catalysts need not necessarily comprise NiO in order to be able to perform the direct amination of hydrocarbons described here in accordance with the invention, but catalysts having an NiO content are frequently superior to those without NiO in their performance for the direct amination.

Examples of suitable catalysts which, however, do not restrict the scope of the invention have already been described in the literature.

For example, suitable catalysts according to (a), whose catalytically active composition comprises from 20 to 85% by weight of oxygen compounds of zirconium, calculated as ZrO₂, from 1 to 30% by weight of oxygen compounds of copper, calculated as CuO, from 30 to 70% by weight of oxygen compounds of nickel, calculated as NiO, from 0.1 to 5% by weight of oxygen compounds of molybdenum, calculated as MoO₃, and from 0 to 10% by weight of oxygen compounds of aluminum and/or of manganese, calculated as Al₂O₃ and MnO₂ respectively, are described, inter alia, in DE-A 44 28 004 (see Example 1).

For example, suitable catalysts according to (b), whose catalytically active composition comprises from 22 to 45% by weight of oxygen compounds of zirconium, calculated as ZrO₂, from 1 to 30% by weight of oxygen compounds of copper, calculated as CuO, from 5 to 50% by weight of oxygen compounds of nickel, calculated as NiO, from 5 to 50% by weight of oxygen compounds of cobalt, calculated as CoO, from 0 to 5% by weight of oxygen compounds of molybdenum, calculated as MoO₃, and from 0 to 10% by weight of oxygen compounds of aluminum and/or of manganese, calculated as Al₂O₃ and MnO₂ respectively, are described, inter alia, in EP 1 106 600.

EP 963 975 also describes catalysts according to (b); see Example 3.

It is possible with the amination process according to the invention to aminate any hydrocarbons, such as aromatic hydrocarbons, aliphatic hydrocarbons and cycloaliphatic hydrocarbons, which may have any substitution and may have heteroatoms and double or triple bonds within their chain or their ring/their rings. In the amination process according to the invention, preference is given to using aromatic hydrocarbons and heteroaromatic hydrocarbons. The particular products are the corresponding arylamines or heteroarylamines.

In the context of the present invention, an aromatic hydrocarbon is understood to mean an unsaturated cyclic hydrocarbon which has one or more rings and comprises exclusively aromatic C—H bonds. The aromatic hydrocarbon preferably has one or more 5- or 6-membered rings.

A heteroaromatic hydrocarbon is understood to mean those aromatic hydrocarbons in which one or more of the carbon atoms of the aromatic ring is/are replaced by a heteroatom selected from N, O and S.

The aromatic hydrocarbons or the heteroaromatic hydrocarbons may be substituted or unsubstituted. A substituted aromatic or heteroaromatic hydrocarbon is understood to mean compounds in which one or more hydrogen atoms which is/are bonded to a carbon atom or heteroatom of the aromatic ring is/are replaced by another radical. Such radicals are, for example, substituted or unsubstituted alkyl, alkenyl, alkynyl, heteroalkyl, heteroalkenyl, heteroalkynyl, cycloalkyl and/or cycloalkynyl radicals. In addition, the following radicals are possible: halogen, hydroxyl, alkoxy, aryloxy, amino, amido, thio and phosphino. Preferred radicals of the aromatic or heteroaromatic hydrocarbons are selected from C₁₋₆-alkyl, C₁₋₆-alkenyl, C₁₋₆-alkynyl, C₃₋₈-cycloalkyl, C₃₋₈-cycloalkenyl, alkoxy, aryloxy, amino and amido, where C₁₋₆ relates to the number of carbon atoms in the main chain of the alkyl radical, of the alkenyl radical or of the alkynyl radical, and C₃₋₈ to the number of carbon atoms of the cycloalkyl or cycloalkenyl ring. It is also possible that the substituents (radicals) of the substituted aromatic or heteroaromatic hydrocarbon have further substituents.

The number of substituents (radicals) of the aromatic or heteroaromatic hydrocarbon is arbitrary. In a preferred embodiment, the aromatic or heteroaromatic hydrocarbon has, however, at least one hydrogen atom which is bonded directly to a carbon atom or a heteroatom of the aromatic ring. Thus, a 6-membered ring preferably has 5 or fewer substituents (radicals) and a 5-membered ring preferably has 4 or fewer substituents (radicals). A 6-membered aromatic or heteroaromatic ring more preferably bears 4 or fewer substituents, even more preferably 3 or fewer substituents (radicals). A 5-membered aromatic or heteroaromatic ring preferably bears 3 or fewer radicals, more preferably 2 or fewer radicals.

In a particularly preferred embodiment of the process according to the invention, an aromatic or heteroaromatic hydrocarbon of the general formula

(A)-(B)ₙ

is used, where the symbols are each defined as follows:
A is independently aryl or heteroaryl, A is preferably selected from phenyl, diphenyl, diphenylmethane, benzyl, dibenzyl, naphthyl, anthracene, pyridyl and quinoline;
n is from 0 to 5, preferably from 0 to 4, especially in the case when A is a 6-membered aryl or heteroaryl ring; in the case that A is a 5-membered aryl or heteroaryl ring, n is preferably from 0 to 4; irrespective of the ring size, n is more preferably from 0 to 3, most preferably from 0 to 2 and in particular from 0 to 1; the remaining hydrocarbon atoms or heteroatoms of A which do not bear any substituents B bear hydrogen atoms, or, if appropriate, no substituents;
B is independently selected from the group consisting of alkyl, alkenyl, alkynyl, substituted alkyl, substituted alkenyl, substituted alkynyl, heteroalkyl, substituted heteroalkyl, heteroalkenyl, substituted heteroalkenyl, heteroalkynyl, substituted heteroalkynyl, cycloalkyl, cycloalkenyl, substituted cycloalkyl, substituted cycloalkenyl, halogen, hydroxy, alkoxy, aryloxy, carbonyl, amino, amido, thio and phosphino; B is preferably independently selected from C₁₋₆-alkyl, C₁₋₆-alkenyl, C₁₋₆-alkynyl, C₃₋₈-cycloalkyl, C₃₋₈-cycloalkenyl, alkoxy, aryloxy, amino and amido.

The term “independently” means that, when n is 2 or greater, the substituents B may be identical or different radicals from the groups mentioned.

In the present application, alkyl is understood to mean branched or unbranched, saturated acyclic hydrocarbyl radicals. Examples of suitable alkyl radicals are methyl, ethyl, n-propyl, i-propyl, n-butyl, t-butyl, i-butyl, etc. The alkyl radicals used preferably have from 1 to 50 carbon atoms, more preferably from 1 to 20 carbon atoms, even more preferably from 1 to 6 carbon atoms and in particular from 1 to 3 carbon atoms.

In the present application, alkenyl is understood to mean branched or unbranched, acyclic hydrocarbyl radicals which have at least one carbon-carbon double bond. Suitable alkenyl radicals are, for example, 2-propenyl, vinyl, etc. The alkenyl radicals have preferably from 2 to 50 carbon atoms, more preferably from 2 to 20 carbon atoms, even more preferably from 2 to 6 carbon atoms and in particular from 2 to 3 carbon atoms. The term alkenyl also encompasses radicals which have either a cis-orientation or a trans-orientation (alternatively E or Z orientation).

In the present application, alkynyl is understood to mean branched or unbranched, acyclic hydrocarbyl radicals which have at least one carbon-carbon triple bond. The alkynyl radicals preferably have from 2 to 50 carbon atoms, more preferably from 2 to 20 carbon atoms, even more preferably from 1 to 6 carbon atoms and in particular from 2 to 3 carbon atoms.

Substituted alkyl, substituted alkenyl and substituted alkynyl are understood to mean alkyl, alkenyl and alkynyl radicals in which one or more hydrogen atoms which are bonded to one carbon atom of these radicals are replaced by another group. Examples of such other groups are heteroatoms, halogen, aryl, substituted aryl, cycloalkyl, cycloalkenyl, substituted cycloalkyl, substituted cycloalkenyl and combinations thereof. Examples of suitable substituted alkyl radicals are benzyl, trifluoromethyl, inter alia.

The terms heteroalkyl, heteroalkenyl and heteroalkynyl are understood to mean alkyl, alkenyl and alkynyl radicals in which one or more of the carbon atoms in the carbon chain is replaced by a heteroatom selected from N, O and S. The bond between the heteroatom and a further carbon atom may be saturated, or, if appropriate, unsaturated.

In the present application, cycloalkyl is understood to mean saturated cyclic nonaromatic hydrocarbyl radicals which are composed of a single ring or a plurality of fused rings. Suitable cycloalkyl radicals are, for example, cyclopentyl, cyclohexyl, cyclooctanyl, bicyclooctyl, etc. The cycloalkyl radicals have preferably between 3 and 50 carbon atoms, more preferably between 3 and 20 carbon atoms, even more preferably between 3 and 8 carbon atoms and in particular between 3 and 6 carbon atoms.

In the present application, cycloalkenyl is understood to mean partly unsaturated, cyclic nonaromatic hydrocarbyl radicals which have a single fused ring or a plurality of fused rings. Suitable cycloalkenyl radicals are, for example, cyclopentenyl, cyclohexenyl, cyclooctenyl, etc. The cycloalkenyl radicals have preferably from 3 to 50 carbon atoms, more preferably from 3 to 20 carbon atoms, even more preferably from 3 to 8 carbon atoms and in particular from 3 to 6 carbon atoms.

Substituted cycloalkyl and substituted cycloalkenyl radicals are cycloalkyl and cycloalkenyl radicals, in which one or more hydrogen atoms of any carbon atom of the carbon ring is replaced by another group. Such other groups are, for example, halogen, alkyl, alkenyl, alkynyl, substituted alkyl, substituted alkenyl, substituted alkynyl, aryl, substituted aryl, cycloalkyl, cycloalkenyl, substituted cycloalkyl, substituted cycloalkenyl, an aliphatic heterocyclic radical, a substituted aliphatic heterocyclic radical, heteroaryl, substituted heteroaryl, alkoxy, aryloxy, boryl, phosphino, amino, silyl, thio, seleno and combinations thereof. Examples of substituted cycloalkyl and cycloalkenyl radicals are 4-dimethylaminocyclohexyl, 4,5-dibromocyclohept-4-enyl, inter alia.

In the context of the present application, aryl is understood to mean aromatic radicals which have a single aromatic ring or a plurality of aromatic rings which are fused, joined via a covalent bond or joined by a suitable unit, for example a methylene or ethylene unit. Such suitable units may also be carbonyl units, as in benzophenol, or oxygen units, as in diphenyl ether, or nitrogen units, as in diphenylamine. The aromatic ring or the aromatic rings are, for example, phenyl, naphthyl, diphenyl, diphenyl ether, diphenylamine and benzophenone. The aryl radicals preferably have from 6 to 50 carbon atoms, more preferably from 6 to 20 carbon atoms, most preferably from 6 to 8 carbon atoms.

Substituted aryl radicals are aryl radicals in which one or more hydrogen atoms which are bonded to carbon atoms of the aryl radical are replaced by one or more other groups. Suitable other groups are alkyl, alkenyl, alkynyl, substituted alkyl, substituted alkenyl, substituted alkynyl, cycloalkyl, cycloalkenyl, substituted cycloalkyl, substituted cycloalkenyl, heterocyclo, substituted heterocyclo, halogen, halogen-substituted alkyl (e.g. CF₃), hydroxyl, amino, phosphino, alkoxy, thio and both saturated and unsaturated cyclic hydrocarbons which may be fused on the aromatic ring or on the aromatic rings or may be joined by a bond, or may be joined to one another via a suitable group. Suitable groups have already been mentioned above.

According to the present application, heterocyclo is understood to mean a saturated, partly unsaturated or unsaturated, cyclic radical in which one or more carbon atoms of the radical are replaced by a heteroatom, for example N, O or S. Examples of heterocyclo radicals are piperazinyl, morpholinyl, tetrahydropyranyl, tetrahydrofuranyl, piperidinyl, pyrrolidinyl, oxazolinyl, pyridyl, pyrazyl, pyridazyl, pyrimidyl.

Substituted heterocyclo radicals are those heterocyclo radicals in which one or more hydrogen atoms which are bonded to one of the ring atoms are replaced by another group. Suitable other groups are halogen, alkyl, substituted alkyl, aryl, substituted aryl, heteroaryl, substituted heteroaryl, alkoxy, aryloxy, boryl, phosphino, amino, silyl, thio, seleno and combinations thereof.

Alkoxy radicals are understood to mean radicals of the general formula —OZ¹ in which Z¹ is selected from alkyl, substituted alkyl, cycloalkyl, substituted cycloalkyl, heterocycloalkyl, substituted heterocycloalkyl, silyl and combinations thereof. Suitable alkoxy radicals are, for example, methoxy, ethoxy, benzyloxy, t-butoxy, etc. The term aryloxy is understood to mean those radicals of the general formula —OZ¹ in which Z¹ is selected from aryl, substituted aryl, heteroaryl, substituted heteroaryl and combinations thereof. Suitable aryloxy radicals are phenoxy, substituted phenoxy, 2-pyridinoxy, 8-quinolinoxy, inter alia.

Amino radicals are understood to mean radicals of the general formula —NZ¹Z² in which Z¹ and Z² are each independently selected from hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof.

Aromatic or heteroaromatic hydrocarbons used with preference in the amination process according to the invention are selected from benzene, diphenylmethane, naphthalene, anthracene, toluene, xylene, phenol and aniline, and also pyridine, pyrazine, pyridazine, pyrimidine and quinoline. It is also possible to use mixtures of the aromatic or heteroaromatic hydrocarbons mentioned. Particular preference is given to using the aromatic hydrocarbons, benzene, naphthalene, anthracene, toluene, xylene, pyridine, phenol and/or aniline, very particular preference to using benzene, toluene and/or pyridine.

Especially preferably, benzene is used in the amination process according to the invention, so that the product formed is aniline.

The compound through which the amino group is introduced is more preferably ammonia. This means that, in accordance with the invention, the hydrocarbons, especially the benzene, are more preferably reacted with ammonia. If appropriate, compounds which eliminate ammonia under the reaction conditions may also find use.

For the preparation of mono- and dialkyl-N,(N)-substituted aromatic amines, for example mono- and/or dimethylaniline, it is also possible to use mono- and dialkylamines, preferably mono- and di(m)ethylamine.

The reaction conditions in the amination processes according to the invention are dependent upon factors including the aromatic hydrocarbon to be aminated and the catalyst used.

The amination, preferably the amination of benzene, i.e. the reaction of benzene with ammonia, is effected generally at temperatures of from 200 to 800° C., preferably from 300 to 500° C., more preferably from 350 to 400° C. and most preferably from 350 to 500° C.

The reaction pressure in the amination, preferably in the amination of benzene, i.e. the reaction of benzene with ammonia, is preferably from 1 to 1000 bar, more preferably from 2 to 300 bar, in particular from 2 to 150 bar, especially preferably from 15 to 110 bar.

The residence time in the amination process according to the invention, preferably in the amination of benzene, in the case of performance in a batchwise process, is generally from 15 minutes to 8 hours, preferably from 15 minutes to 4 hours, more preferably from 15 minutes to 1 hour. In the case of performance in a preferred continuous process, the residence time is generally from 0.1 second to 20 minutes, preferably from 0.5 second to 10 minutes. For the preferred continuous processes, “residence time” in this context means the residence time over the catalyst, hence the residence time in the catalyst bed for fixed bed catalyst; for fluidized bed reactors, the synthesis part of the reactor (part of the reactor where the catalyst is localized) is considered.

The relative amount of the hydrocarbon used and of the amine component is dependent upon the amination reaction carried out and the reaction conditions. In general, at least stoichiometric amounts of the hydrocarbon and the amine component are used. However, it is typically preferred to use one of the reaction partners in a stoichiometric excess in order to achieve a shift in the equilibrium to the side of the desired product and hence a higher conversion. Preference is given to using the amine component in a stoichiometric excess.

The amination process according to the invention may be carried out continuously, batchwise or semicontinuously. Suitable reactors are thus both stirred tank reactors and tubular reactors. Typical reactors are, for example, high pressure stirred tank reactors, autoclaves, fixed bed reactors, fluidized bed reactors, moving beds, circulating fluidized beds, salt bath reactors, plate heat exchangers as reactors, tray reactors having a plurality of trays with or without heat exchange or drawing/feeding of substreams between the trays, in possible designs as radial flow or axial flow reactors, continuous stirred tanks, bubble reactors, etc., and the reactor suitable in each case for the desired reaction conditions (such as temperature, pressure and residence time) is used. The reactors may each be used as a single reactor, as a series of individual reactors and/or in the form of two or more parallel reactors. The reactors may be operated in an AB mode (alternating mode). The process according to the invention may be carried out as a batch reaction, semicontinuous reaction or continuous reaction. The specific reactor construction and performance of the reaction may vary depending on the amination process to be carried out, the state of matter of the aromatic hydrocarbon to be aminated, the required reaction times and the nature of the catalyst used. Preference is given to carrying out the process according to the invention for direct amination in a high pressure stirred tank reactor, fixed bed reactor or fluidized bed reactor.

In a particularly preferred embodiment, one or more fixed bed reactors are used in the amination of benzene to aniline.

The hydrocarbon and the amine component may be introduced in gaseous or liquid form into the reaction zone of the particular reactor. The preferred phase is dependent in each case upon the amination carried out and the reactor used. In a preferred embodiment, for example in the preparation of aniline from benzene, benzene and ammonia are preferably present as gaseous reactants in the reaction zone. Typically, benzene is fed as a liquid which is heated and evaporated to form a gas, while ammonia is present either in gaseous form or in a supercritical phase in the reaction zone. It is likewise possible that benzene is present in a supercritical phase at least together with ammonia.

The hydrocarbon and the amine component may be introduced together into the reaction zone of the reactor, for example as a premixed reactant stream, or separately. In the case of a separate addition, the hydrocarbon and the amine component may be introduced simultaneously, offset in time or successively into the reaction zone of the reactor. Preference is given to adding the amine component and adding the hydrocarbon offset in time.

If appropriate, further coreactants, cocatalysts or further reagents are introduced into the reaction zone of the reactor in the process according to the invention, depending in each case on the amination carried out. For example, in the amination of benzene, oxygen or an oxygen-comprising gas may be introduced into the reaction zone of the reactor as a coreactant. The relative amount of gaseous oxygen which can be introduced into the reaction zone is variable and depends upon factors including the catalyst system used. The molar ratio of gaseous oxygen to aniline may, for example, be in the range from 0.05:1 to 1:1, preferably from 0.1:1 to 0.5:1. However, it is also possible to perform the amination of benzene without addition of oxygen or an oxygen-comprising gas into the reaction zone.

The amination can be performed preferably at a molar ratio of ammonia to hydrocarbon of at least 1.

After the amination, the desired product can be isolated by processes known to those skilled in the art.

### EXAMPLES

### Example 1

### Preparation of Catalyst

The catalyst is prepared in accordance with DE-A 44 28 004:

An aqueous solution of nickel nitrate, copper nitrate and zirconium acetate which comprises 4.48% by weight of Ni (calculated as NiO), 1.52% by weight of Cu (calculated as CuO) and 2.82% by weight of Zr (calculated as ZrO₂) is precipitated simultaneously in a stirrer vessel in a constant stream with a 20% aqueous sodium carbonate solution at a temperature of 70° C., in such a way that the pH of 7.0 measured with a glass electrode is maintained. The resulting suspension is filtered and the filtercake is washed with mineralized water until the electrical conductivity of the filtrate is approx. 20 μS. Sufficient ammonium heptamolybdate is then incorporated into the still-moist filtercake that the oxide mixture specified below is obtained. Thereafter, the filtercake is dried at a temperature of 150° C. in a drying cabinet or a spray dryer. The hydroxide-carbonate mixture obtained in this way is then heat-treated at a temperature of from 430 to 460° C. over a period of 4 hours. The oxidic species thus prepared has the composition: 50% by weight of NiO, 17% by weight of CuO, 1.5% by weight of MoO₃ and 31.5% by weight of ZrO₂. The catalyst was mixed with 3% by weight of graphite and shaped to tablets.

### Example 2

### Preparation of Catalyst

An aqueous solution of nickel nitrate, copper nitrate, magnesium nitrate and aluminum nitrate which comprises 8.1 kg of NiO, 2.9 kg of CuO, 2.8 kg of MgO and 10.2 kg of Al₂O₃ in 111 kg of total solution is precipitated simultaneously in a stirred vessel in a constant stream with an aqueous solution of 7.75 kg of sodium carbonate and 78 kg of sodium hydroxide in a total volume of 244 liters at a temperature of 20° C., in such a way that the pH of 9.5 measured with a glass electrode is maintained. The resultant suspension is filtered and the filtercake is washed with the demineralized water until the electrical conductivity of the filtrate is approx. 20 μS. Thereafter, the filtercake is dried in a drying cabinet at a temperature of 150° C. The hydroxide-carbonate mixture obtained in this way is then heat-treated at a temperature of from 430 to 460° C. over a period of 4 hours. The oxidic species thus prepared has the composition: 56.6% by weight of NiO, 19.6% by weight of CuO, 15.4% by weight of MgO and 8.5% by weight of Al₂O₃.

### Example 3

### Preparation of Catalyst (Based on Ni—Co—Cu/ZrO₂ According to EP-A-963 975)

An aqueous solution of nickel nitrate, cobalt nitrate, copper nitrate and zirconium acetate, which comprised 2.39% by weight of NiO, 2.39% by weight of CoO, 0.94% by weight of CuO and 2.82% by weight of ZrO₂, was precipitated simultaneously in a stirred vessel in a constant stream with a 20% aqueous sodium carbonate solution at a temperature of 70° C. such that the pH of 7.0 measured with a glass electrode was maintained. The resulting suspension was filtered and the filtercake was washed with demineralized water until the electrical conductivity of the filtrate was approx. 20 μS. Thereafter, the filtercake was dried at a temperature of 150° C. in a drying cabinet or a spray-dryer. The hydroxide-carbonate mixture obtained in this way was then heat-treated at a temperature of from 450 to 500° C. over a period of 4 hours. The catalyst thus prepared had the composition: 28% by weight of NiO, 28% by weight of CoO, 11% by weight of CuO and 33% by weight of ZrO₂. The catalyst was mixed with 3% by weight of graphite and shaped to tablets. The oxidic tablets were reduced. The reduction was performed at 280° C., in the course of which the heating rate was 3° C./minute. Reduction was effected first with 10% H₂ in N₂ for 50 minutes, then with 25% H₂ in N₂ for 20 minutes, then with 50% H₂ in N₂ for 10 minutes, then with 75% H₂ in N₂ for 10 minutes and finally with 100% H₂ for 3 hours. The percentages are each % by volume. The passivation of the reduced catalyst was performed at room temperature in dilute air (air in N₂ with a maximum O₂ content of 5% by volume).

### Example 4

### Amination of Benzene on Catalyst without Addition of Nitrobenzene (Comparative Example)

A tubular reactor charged with 2-4 mm quartz glass spall at the reactor inlet, 20 ml=23.6 g of catalyst from Example 1 in the form of 6×3 mm tablets and 2-4 mm of quartz glass spall at the reactor outlet is heated to 350° C. under air (50 l (STP)/h). After the heating, the air supply is stopped, the reactor is purged with nitrogen, and then the feed is started up. At a total pressure of 85 bar and an internal reactor temperature of 350° C., 59.4 g of benzene/hour and 118 g of ammonia/hour are supplied to the catalyst. The effluent from the reactor is cooled to a temperature of 2° C., and the condensate is homogenized with methanol and analyzed by means of gas chromatography with an internal standard. The aniline yield in a collected sample for which the collection period was started after 3.5 h of running time and ended after 4 h, was 8.2 mmol of aniline/mole of benzene supplied and hour. This corresponds to a space-time yield of 28.89 g of aniline/liter of catalyst and hour.

An online gas chromatography sample of the offgas, which consisted of ammonia in particular, showed a hydrogen content of 0.128% by volume in the offgas after an experiment running time of 4.0 h, which corresponds to hydrogen formation of 11 mmol of hydrogen/mole of benzene supplied and hour.

The amount of hydrogen in the offgas rises continuously with the running time and with increasing reduction of the catalyst: after 1.4 h, it is 3 mmol of H2/mole of benzene supplied and hour, after 2.8 h 8 mmol of H2/mole of benzene supplied and hour, after 4 h 11 mmol of H2/mole of benzene supplied and hour, and after 4.6 h 14 mmol of H2/mole of benzene supplied and hour.

### Example 5 (Inventive)

### Amination of Benzene on Catalyst with Addition of 0.5% Nitrobenzene in the Benzene Feed

A tubular reactor charged with 2-4 mm quartz glass spall at the reactor inlet, 20 ml=23.6 g of catalyst from Example 1 in the form of 6×3 mm tablets and 2-4 mm of quartz glass spall at the reactor outlet is heated to 350° C. under air (50 l (STP)/h). After the heating, the air supply is stopped, the reactor is purged with nitrogen, and then the feed is started up. At a total pressure of 85 bar and an internal reactor temperature of 350° C., 59.6 g of an aromatics mixture consisting of 99.5% benzene and 0.5% nitrobenzene (i.e. 0.3 g of nitrobenzene/h and 0.002 mol of nitrobenzene/h respectively)/hour and 118 g of ammonia/hour are supplied to the catalyst. The effluent from the reactor is cooled to a temperature of 2° C., and the condensate is homogenized with methanol and analyzed by means of gas chromatography with an internal standard. The aniline yield in a collected sample for which the collection period was started after 3.5 h of running time and ended after 4 h, was 11.3 mmol of aniline/mole of aromatics supplied and hour. This corresponds to a space-time yield of 40.15 g of aniline/liter of catalyst and hour.

An online gas chromatography sample of the offgas, which consisted of ammonia in particular, showed a hydrogen content of 0.034% by volume in the offgas after an experiment running time of 4.0 h, which corresponds to hydrogen formation of 4 mmol of hydrogen/mole of benzene supplied and hour.

In this example too, the amount of hydrogen in the offgas rises again with the running time and with increasing reduction of the catalyst, but to a significantly lower absolute level and more slowly than in Example 3: after 1.2 h, it is 1 mmol of H2/mole of aromatics supplied and hour, after 2.1 h 2 mmol of H2/mole of aromatics supplied and hour, after 4 h 4 mmol of H2/mole of aromatics supplied and hour, and after 4.8 h 5 mmol of H2/mole of aromatics supplied and hour.

### Example 6 (Inventive)

### Amination of Benzene on Catalyst with Addition of 1.0% Nitrobenzene in the Benzene Feed

A tubular reactor charged with 2-4 mm quartz glass spall at the reactor inlet, 20 ml=23.6 g of catalyst from Example 1 in the form of 6×3 mm tablets and 2-4 mm of quartz glass spall at the reactor outlet is heated to 350° C. under air (50 l (STP)/h). After the heating, the air supply is stopped, the reactor is purged with nitrogen, and then the feed is started up. At a total pressure of 85 bar and an internal reactor temperature of 350° C., 59.1 g of an aromatics mixture consisting of 99.0% benzene and 1.0% nitrobenzene (i.e. 0.6 g of nitrobenzene/h and 0.005 mol of nitrobenzene/h respectively)/hour and 118 g of ammonia/hour are supplied to the catalyst. The effluent from the reactor is cooled to a temperature of 2° C., and the condensate is homogenized with methanol and analyzed by means of gas chromatography with an internal standard. The aniline yield in a collected sample for which the collection period was started after 3.5 h of running time and ended after 4 h, was 17.8 mmol of aniline/mole of aromatics supplied and hour. This corresponds to a space-time yield of 62.93 g of aniline/liter of catalyst and hour.

An online gas chromatography sample of the offgas, which consisted of ammonia in particular, showed a hydrogen content of 0.025% by volume in the offgas after an experiment running time of 4.0 h, which corresponds to hydrogen formation of 2 mmol of hydrogen/mole of benzene supplied and hour.

In this example too, the amount of hydrogen in the offgas rises again with the running time and with increasing reduction of the catalyst, but to a significantly lower absolute level and more slowly than in Example 4: after 1.0 h, it is 1 mmol of H2/mole of aromatics supplied and hour, after 2.1 h 1 mmol of H2/mole of aromatics supplied and hour, after 3.0 h 2 mmol of H2/mole of aromatics supplied and hour, after 4 h 2 mmol of H2/mole of aromatics supplied and hour, and after 5.0 h 3 mmol of H2/mole of aromatics supplied and hour.

### Example 7 (Inventive)

### Amination of Benzene on Catalyst with Addition of 3.0% Nitrobenzene in the Benzene Feed

A tubular reactor charged with 2-4 mm quartz glass spall at the reactor inlet, 20 ml=23.6 g of catalyst from Example 1 in the form of 6×3 mm tablets and 2-4 mm of quartz glass spall at the reactor outlet is heated to 350° C. under air (50 l (STP)/h). After the heating, the air supply is stopped, the reactor is purged with nitrogen, and then the feed is started up. At a total pressure of 85 bar and an internal reactor temperature of 350° C., 60.1 g of an aromatics mixture consisting of 97% benzene and 3% nitrobenzene (i.e. 1.858 g of nitrobenzene/h and 0.015 mol of nitrobenzene/h respectively)/hour and 118 g of ammonia/hour are supplied to the catalyst. The effluent from the reactor is cooled to a temperature of 2° C., and the condensate is homogenized with methanol and analyzed by means of gas chromatography with an internal standard. The aniline yield in a collected sample for which the collection period was started after 3.5 h of running time and ended after 4 h, was 12.2 mmol of aniline/mole of aromatics supplied and hour. This corresponds to a space-time yield of 44.39 g of aniline/liter of catalyst and hour. In this experiment, the peak value was attained as early as after 1 h and was 15.1 mmol of aniline/mole of aromatics supplied and hour. This corresponds to a space-time yield of 55.24 g of aniline/liter of catalyst and hour.

All online gas chromatography samples of the offgas of this experiment, which consisted of ammonia in particular, showed a hydrogen content below the limit of detection of the GC instrument, i.e. approx. <30-50 ppm. Even after a running time of 5 h, no hydrogen could be detected.

### Example 8 (Inventive)

### Amination of Benzene on Catalyst with Addition of 11.1% Nitrobenzene in the Benzene Feed

A tubular reactor charged with 2-4 mm quartz glass spall at the reactor inlet, 20 ml=23.6 g of catalyst from Example 1 in the form of 6×3 mm tablets and 2-4 mm of quartz glass spall at the reactor outlet is heated to 350° C. under air (50 l (STP)/h). After the heating, the air supply is stopped, the reactor is purged with nitrogen, and then the feed is started up. At a total pressure of 85 bar and an internal reactor temperature of 350° C., 61.3 g of an aromatics mixture consisting of 88.9% benzene and 11.1% nitrobenzene (i.e. 6.8 g of nitrobenzene/h and 0.055 mol of nitrobenzene/h respectively)/hour and 118 g of ammonia/hour are supplied to the catalyst. The effluent from the reactor is cooled to a temperature of 2° C., and the condensate is homogenized with methanol and analyzed by means of gas chromatography with an internal standard. The aniline yield in a collected sample for which the collection period was started after 3.5 h of running time and ended after 4 h, was 30.9 mmol of aniline/mole of aromatics supplied and hour. This corresponds to a space-time yield of 120.78 g of aniline/liter of catalyst and hour. In this experiment, the peak value was attained as early as after 1 h and was 43.8 mmol of aniline/mole of aromatics supplied and hour. This corresponds to a space-time yield of 171.36 g of aniline/liter of catalyst and hour.

All online gas chromatography samples of the offgas of this experiment, which consisted of ammonia in particular, showed a hydrogen content below the limit of detection of the GC instrument, i.e. approx. <30-50 ppm. Even after a running time of 5 h, no hydrogen could be detected.

1. A process for aminating hydrocarbons with ammonia, which comprises performing the amination in the presence of an additive which reacts with hydrogen, the additive used being at least one organic chemical compound, N₂O, hydroxylamine, hydrazine and/or carbon monoxide.

2. The process according to claim 1, wherein the additive used which reacts with hydrogen is carbon monoxide, carbonyl compounds, nitriles, imines, amides, nitro compounds, nitroso compounds, olefins, alkynes, organic peroxides, organic acids, organic acid derivatives, hydrazine derivatives, hydroxylamines, quinones, aromatics and/or molecules with sp2-hybridized carbon atoms.

3. The process according to claim 1, wherein the additive used which reacts with hydrogen is nitrobenzene, carbon monoxide, hydrocyanic acid, acetonitrile, propionitrile, butyronitrile, benzonitrile, imines from the reaction of benzaldehyde with ammonia or primary amines, imines from the reaction of aliphatic aldehydes with ammonia or primary amines, formamide, acetamide, benzamide, nitrosobenzene, ethene, propene, 1-butene, 2-butene, isobutene, n-pentene and pentene isomers, cyclopentene, n-hexene, hexene isomers, cyclohexene, n-heptene, heptene isomers, cycloheptene, n-octene, octadienes, cyclooctene, cyclooctadiene, acetylene, propyne, butyne, phenylacetylene, meta-chloroperbenzoic acid, formic acid, acetic acid, propionic acid, butyric acid, valeric acid, caproic acid, oxalic acid, malonic acid, succinic acid, maleic acid, fumaric acid, esters or anhydrides of the carboxylic acids mentioned, hydrazine, phenylhydrazine, hydrazides of aliphatic or aromatic ketones, hydroxylamine, alkylhydroxylamines and/or arylhydroxylamine.

4. The process according to claim 1, wherein the additive used which reacts with hydrogen is reducible nitrogen compounds, acetylene, alkynes having from 3 to 6 carbon atoms and/or olefins having from 2 to 6 carbon atoms.

5. The process according to claim 1, wherein the additive used which reacts with hydrogen is nitrobenzene.

6. The process according to claim 1, wherein the additive used which reacts with hydrogen is hydrazine.

7. The process according to claim 1, wherein the additive used which reacts with hydrogen is hydroxylamine.

8. The process according to claim 1, wherein the additive used which reacts with hydrogen is N₂O.

9. The process according to claim 1, wherein benzene is reacted with ammonia in the presence of catalyst which catalyzes the amination and the additive used which reacts with hydrogen is nitrobenzene.

10. The process according to claim 1, wherein the additive which reacts with hydrogen is metered in together with the hydrocarbon at the inlet of the reactor.

11. The process according to claim 1, wherein a nitrobenzene/benzene mixture is introduced into the reactor in a common feed line and ammonia in another feed line, in each case at the inlet of the reactor.

12. The process according to claim 1, wherein a nitrobenzene/benzene mixture from a common feed line and the ammonia from a second feed line are first combined in a mixer or evaporator and introduced as a homogeneous mixture onto a catalyst bed which catalyzes the amination.

13. The process according to claim 1, wherein the proportion by weight of the additive which reacts with hydrogen is between 0.001% by weight and 50% by weight based on the total weight of the hydrocarbon used and the additive.

14. The process according to claim 1, wherein a mixture comprising benzene and nitrobenzene is used which comprises between 0.1% by weight and 15% by weight of nitrobenzene based on the total weight of nitrobenzene and benzene.

15. The process according to claim 1, wherein a catalyst is used which catalyzes both the direct amination of the hydrocarbon and the hydrogenation of the additive.

16. The process according to claim 1, wherein the catalysts used are compounds which comprise Ni—Cu—X, Fe—Cu—X, Ni—Cu—Co—X and/or Co—Cu—X, where X is Ag or Mo.

17. The process according to claim 16, wherein the proportion by weight of the elements Ni, Co and Fe together in the catalyst is between 0.1% by weight and 75% by weight, and the proportion by weight of Cu is between 0.1% by weight and 75% by weight, based in each case on the total weight of the catalyst.

18. The process according to claim 16, wherein the proportion by weight of the doping element X in the total weight of the catalyst is between 0.01% by weight and 8% by weight based on the total weight of the catalyst.

19. The process according to claim 1, wherein the catalyst used is NiO, CuO and/or MoO₃ on zirconium oxide as a support.

20. The process according to claim 1, wherein the catalyst used is NiO, CuO and/or CoO on zirconium oxide as a support.

21. The process according to claim 1, wherein the catalyst used is NiO, CuO and/or Fe₂O₃ on zirconium oxide as a support.

22. The process according to claim 1, wherein the catalyst used is NiO, CuO and/or FE₂O₃ on magnesium aluminum oxide as a support.

23. The process according to claim 1, wherein the catalyst used is at least one of the following compounds (a), (b), (c) and/or (d):
(a) comprising, preferably consisting of, NiO, CuO and MoO₃, on zirconium oxide as a support,
(b) (preferably mainly) comprising NiO, CuO and CoO on zirconium oxide as a support,
(c) comprising, preferably consisting of, NiO and/or CuO and/or Fe₂O₃ on zirconium oxide as a support,
(d) comprising, preferably consisting of, NiO and/or CuO and/or Fe₂O₃ on magnesium aluminum oxide as a support,
catalysts (a) to (d) being usable individually or in combination with one another.

24. The process according to claim 1, wherein the amination is performed continuously.

25. The process according to claim 1, wherein the amination is performed at temperatures between 200 and 800° C.

26. The process according to claim 1, wherein the amination is performed at pressures between 1 and 1000 bar.
